(19) United States
(12) Patent Application Publication
(10) Pub. No.: US 2025/0020652 A1
(43) Pub. Date: Jan. 16, 2025

(54) GRANULOCYTE-MACROPHAGE COLONY-STIMULATING FACTOR-BASED CANCER TREATMENTS

(71) Applicant: PARTNER THERAPEUTICS, INC., Lexington, MA (US)

(72) Inventors: Ila JOSHI, Lexington, MA (US); Debasish ROYCHOWDHURY, Lexington, MA (US)

(21) Appl. No.: 18/710,699

(22) PCT Filed: Nov. 18, 2022
(86) PCT No.: PCT/US2022/080105

§ 371 Date: May 16, 2024

Related U.S. Application Data
(60) Provisional application No. 63/280,737, filed on Nov. 18, 2021.

(51) Int. Cl. G01N 33/574; A61K 38/19; A61K 45/06

(52) U.S. Cl. CPC G01N 33/57484; A61K 38/193; A61K 45/06; G01N 2800/52

(57) ABSTRACT

The present disclosure relates to the treatment of cancer with granulocyte-macrophage colony-stimulating factor and checkpoint inhibitor agents.

### CROSS-REFERENCE TO RELATED APPLICATIONS

This application claims priority to and the benefit of U.S. Provisional Patent Application No. 63/280,737, filed on Nov. 18, 2021, the entire content of which is hereby incorporated herein by reference in its entirety.

### FIELD

This disclosure relates to, in part, treatment and/or mitigation of cancer by modulating immune cells, as well as diagnostic, prognostic and subject selection methods.

### SEQUENCE LISTING

The contents of the text file submitted electronically herewith are incorporated herein by reference in their entirety: A computer readable format copy of the Sequence Listing (filename: “Sequence_Listing_PNR-006PC_127114-5006_SEQ.XML”; date recorded: Nov. 18, 2022; file size: 2,887 bytes).

### BACKGROUND

According to the World Health Organization, cancer is a global pandemic that causes nearly 7 million deaths each year worldwide. That number is expected to reach 10 million by the year 2020. Traditionally, cancer is treated using a variety of modalities including surgery, radiation therapy, and chemotherapy. The choice of treatment depends upon the type, location, and dissemination of the cancer. However, these modalities have proven to be relatively ineffective. The interaction between cancer and the immune system is complex and multifaceted. See de Visser et al., Nat. Rev. Cancer. 2006. 6:24-37. While many cancer patients appear to develop an anti-tumor immune response, cancers also develop strategies to evade immune detection and destruction. Recently, immunotherapy has been developed for the treatment and prevention of cancer and other disorders. Particularly, immunotherapy provides the advantage of cell specificity that other treatment modalities lack. As such, methods for enhancing the efficacy of immune based therapies can be clinically beneficial.

Advances in defining the mechanisms and molecules that regulate immune response have provided novel therapeutic targets for treating cancer. For example, costimulatory and coinhibitory molecules play a central role in the regulation of T cell immune response. Programmed cell death protein-1 (PD-1), like cytotoxic T lymphocyte associated antigen 4 (CTLA-4), is a coinhibitory receptor that is expressed by all T cells during activation. It regulates T cell effector functions during various physiological responses, including acute and chronic infection, cancer and autoimmunity, and in immune homeostasis. In addition to being expressed by conventional T cells, PD-1 is expressed by regulatory T cells, B cells, natural killer cells and some myeloid cell populations. However, compared with conventional T cells, less is known about how PD-1 inhibitory signals regulate these cell types. PD-1's ligand, Programmed cell death 1 ligand-1 (PD-L1) shows broad expression on both hematopoietic and non-hematopoietic cells, positioning the PD-1 pathway as a key regulator of immune cell functions in both secondary lymphoid organs and in non-lymphoid tissues.

The PD-1/PD-L1 axis protects normal tissues from immune attack during episodes of local inflammation by promoting T cell tolerance. PD-1 is more widely expressed than CTLA-4 and can be detected on activated T cells, B cells and NK cells, and over a longer time frame than CTLA-4 (6-12 hours for PD-1 compared with 1 hour for CTLA-4). PD-1 binds programmed death ligand 1 and 2, while PD-L1 also interacts with CD80 and PD-L2 interacts with RGMb (repulsive guidance molecule B). PD-L1 is expressed by tumor cells and immune cells, while PD-L2 is only expressed on dendritic cells in normal tissue. All these interactions transmit an inhibitory signal. After binding, PD-1 becomes clustered with T cell receptors (TCRs) and recruits phosphatase SHP2 (Src homology 2 domain-containing tyrosine phosphatase 2) via its immunoreceptor tyrosine-based switch motif, which induces dephosphorylation of the proximal TCR signaling molecules and suppression of T cell activation. Exhausted T cells have been shown to express various inhibitory receptors (PD-1, CTLA-4, Tim-3, TIGIT and LAG-3) and their pattern of expression (frequency and level) correlates with different levels of exhaustion. The results of a recent study challenge this dogma by showing that CD28 is the most sensitive target for dephosphorylation by the PD-1-Shp2 complex. See Yokosuka T et al. J Exp Med 2012; 209:1201-17; Xiao Y et al. J Exp Med 2014; 211:943-59; Topalian S L et al. Cancer Cell 2015; 27:450-61; Hui E et al. Science 2017; 355:1428-33; Granier C et al. ESMO Open. 2017. 2 (2): e000213.

Recently, monoclonal antibodies against PD-1 (nivolumab, Bristol-Myers Squibb, USA; pembrolizumab, Merck, USA) and PD-L1 (atezolizumab, Genentech, USA; avelumab, EMD Serono, USA and durvalumab, AstraZeneca, UK) have been approved for therapeutic use in various cancers, including melanoma, non-small-cell lung cancer (NSCLC), head and neck squamous cell carcinoma, renal cell carcinoma (RCC), Hodgkin lymphoma, bladder cancer, Merkel cell carcinoma and microsatellite instability high or mismatch repair-deficient adult and pediatric solid tumors. Although understanding of the PD-1 pathway has been translated to therapies that benefit many patients with cancer, most patients do not respond to PD-1 blockade alone (non-responders). Administering treatment to patients without differentiating or selecting those who might be a complete, partial or non-responder can be an inefficient use of resources and could potentially be damaging to the patient since many cancer treatments have significant side effects, such as severe immunosuppression, emesis and/or alopecia. As such, immunotherapy has become a foundational building block for combination therapies aimed at increasing the number of patients who respond, and further work is needed to determine which combinations will work best for which patients. See Page D. et al. Annu. Rev. Med. 2014. 65:185-202; Topalian, S L et al. Cancer Cell. 2015. 27:450-461; Sunshine J et al. 2015. Curr Opin Pharmocol; Sharpe A H and Pauken K E. Nat Rev Immunol. 2018. 18:153-167.

The Human Leukocyte Antigen (HLA) system, also known as the human Major Histocompatibility Complex (MHC) is a cluster of linked genes located on chromosome 6, which is also known as the MHC region. The HLA system is classically divided into three regions: Class I, II, and III regions. See Klein J. In: Gotze D, ed. The Major Histocompatibility System in Man and Animals, New York: Springer-Verlag, 1976:339-378; Choo S Y Yonsei. 2007. 48 (1): 11-23. Class I HLAs comprise a transmembrane protein (heavy chain) and a molecule of beta-2 microglobulin. The class I transmembrane proteins are encoded by the HLA-A, HLA-B and HLA-C loci. A function of class I HLA molecules is to present antigenic peptides (including viral protein antigens) to T cells. Three isoforms of class II MHC molecules, denoted HLA-DR, HLA-DQ, and HLA-DP are currently recognized. The MHC class II molecules have been implicated in the pathogenesis of a number of autoimmune diseases, due to their central roles in the presentation of antigenic peptides to helper T cells. The MHC class II molecules are heterodimers composed of an alpha chain and a beta chain; different alpha- and beta-chains are encoded by subsets of A genes and B genes, respectively. Various Human Leukocyte Antigen-DR (HLA-DR) haplotypes have been recognized and differ in the organization and number of DRB genes present on each DR haplotype; multiple DRB genes have been described. See Bodmer et al., Eur. J. Immunogenetics. 1997. 24:105; Andersson, Frontiers in Bioscience. 1998. 3:739.

The involvement of the HLA system in the development of cancer is still poorly understood. Cancer cells express a number of genes that their normal counterparts do not, and peptides from some of the protein products of these genes bind to HLA molecules. Cancer cells tend to down-regulate the expression of some HLA molecules or stop expressing them altogether, rendering them poor targets for CD8+ cytotoxic T cells. See Klein J and Sato A. N Engl J Med. 2000.343 (20): 1504. This might be one mechanism of tumor immune escape and is often associated with a poor diagnosis in cancer patients.

In addition, it has also been shown that HLA class I and II antigen expression, as well as defects in the antigen processing machinery complex, may predict tumor responses in cancer immunotherapy. In the majority of studies, tsHLA-II (tumor-specific HLA II) molecule expression by cancer cells was associated with better prognosis, improved response to ICI in humans and increased tumor rejection in mouse models of breast cancer, sarcoma, lung cancer and colon cancer. Studies have also demonstrated that (i) reduced tumor HLA class I molecule expression (≤30%) correlated with lack of response to ipilimumab; (ii) HLA class II molecule expression (>1%) correlated with tumor response to nivolumab; and (iii) among nivolumab plus ipilimumab treated patients, reduced HLA class I molecule expression was not associated with progressive disease as well as a decreased tumor response and overall survival. See Rodig S J et al. Sci. Transl. Med. 2018, 10; Axelrod M L. et al. Clin. Cancer Res. 2019. 25:2392-2402; Sabbatino F et al. Int J Mol Sci. 2020 21 (19): 7295. HLA-DR levels in CTLs has been shown to be a highly sensitive and specific potential predictive factor of NACT-response, which can be assessed in blood to guide therapeutic decisions. See Saraiva D P et al. Front. Immunol. 2018. Further, a link between HLA-DR expression and response to anti-PD-1 has been shown, thus suggesting that this IFNg-responsive MHC class II molecule might be a predictive biomarker of anti-PD-1 therapy. See Johnson D B et al. Nat Commun. 2016. 7:10582; Johnson D B et al. Clin Cancer Res. 2018. 24 (21): 5250-5260. In sepsis, functionally impaired monocytes have been characterized by, among other things, having lowered expression of HLA-DR. See Haveman J W et al. Neth J Med. 1999. 55:132-41; Perry S E Int. J. Med. Sci. 2004 1 (3): 126-136.

Granulocyte Macrophage—Colony Stimulating Factor (GM-CSF) is a hematological growth factor that regulates the production, migration, proliferation, differentiation, maturation, activation and function of hematopoietic cells. In response to inflammatory stimuli, GM-CSF is released by various cell types including T lymphocytes, macrophages, fibroblasts and endothelial cells. GM-CSF then activates and enhances the production and survival of neutrophils, eosinophils, and macrophages. Native GM-CSF is usually produced near the site of action where it modulates in vitro proliferation, differentiation, and survival of hematopoietic progenitor cells, but is present in circulating blood in only picomolar concentrations (10⁻¹⁰ to 10⁻¹² M). See Alexander W S. Int Rev Immunol. 1998, 16:651-682; Gasson J C. Blood. 1991, 77:1131-1145; Shannon M F et al. Crit Rev Immunol. 1997, 17:301-323, Barreda D R et al. Dev Comp Immunol. 2004, 28:509-554 and Metcalf D. Immunol Cell Biology. 1987, 65:35-43.

Recombinant human granulocyte-macrophage colony-stimulating factor (rhu GM-CSF) has been approved by the FDA for the treatment of neutropenia, blood dyscrasias and malignancies like leukemia in combination with chemotherapies. In the clinic, GM-CSF used for treatment of neutropenia and aplastic anemia following chemotherapy greatly reduces the risk of infection associated with bone marrow transplantation. Its utility in myeloid leukemia treatment and as a vaccine adjuvant is also well established. See Dorr R T. Clin Therapeutics. 1993. 15 (1): 19-29; Armitage J O. Blood 1998, 92:4491-4508; Kovacic J C et al. J Mol Cell Cardiol. 2007, 42:19-33; Jacobs P P et al. Microbial Cell Factories 2010, 9:93. In vitro studies using a HL-60 (human promyelocytic) cell line to study the effects of GM-CSF on HLA-DR showed that GM-CSF was able to regulate HLA-DR at all levels of expression including gene expression, transcription, surface expression and shedding. See Perry S E Int. J. Med. Sci. 2004 1 (3): 126-136. However, GM-CSF was also shown to license CD14⁺ monocytes such that upon later exposure to INF-γ, the monocytes would switch to an immunosuppressive phenotype through the upregulation of indolamine 2,3-dioxygenase (IDO). This switch to a more suppressive monocyte or suppressive macrophage or monocyte myeloid-derived suppressor cell requires the activation of AKT/mTOR/mTORC1 signaling cascade by GM-CSF. Ribechini E et al. Blood Adv. 2017. 1 (14): 947-960. Thus suggests a dual role of GM-CSF in monocyte functioning.

Combination cancer therapies can potentially increase the percentage of patients who respond to treatment, identify new tumor types that do not respond to monotherapy alone, and improve the quality of clinical responses compared with monotherapy. Hence, there remains a need for new and more effective treatments of cancer utilizing such combination of therapies that can also enhance the host's anti-cancer immune response.

### SUMMARY

Accordingly, in aspects, the present disclosure relates to a method for treating cancer, comprising: administering an effective amount of a composition comprising GM-CSF and an immunotherapeutic agent such as a checkpoint inhibiting agent to a subject in need thereof.

In another aspect, the present disclosure relates to a method for treating cancer, comprising: administering an effective amount of a composition comprising GM-CSF and an immunotherapeutic agent such as a checkpoint inhibiting agent to a subject in need thereof, wherein the subject is characterized by a reduced expression of Human Leukocyte Antigen-DR isotype (HLA-DR).

In still another aspect, the present disclosure provides a method for treating cancer, comprising: (a) identifying a subject undergoing or having undergone treatment with a checkpoint inhibiting agent and presenting as failed, intolerant, resistant, or refractory to the treatment with a checkpoint inhibiting agent; (b) determining the presence, absence or amount of HLA-DR in a sample from the subject; and (c) administering an effective amount of a GM-CSF agent to the subject demonstrating an absence or low level of HLA-DR.

### BRIEF DESCRIPTION OF DRAWINGS

FIG. 1 illustrates graphs depicting the kinetics of expression of HLA-DR induced by LEUKINE. Monocytes and lymphocytes from a human donor were treated with LEUKINE at various concentrations 0.001 nM to 10 nM. Expression of HLA-DR were assessed on day 1, day 2 and day 3 on both monocytes and lymphocytes. In each graph of FIG. 1, the top curve is monocytes, and the bottom curve is lymphocytes.

FIG. 2A illustrates a set of graphs depicting the expression of HLA-DR assessed via flow cytometry on classical monocytes (CD14+CD16+) at the indicated timepoints doses.

FIG. 2B illustrates a graph depicting the HLA-DR expression on classical monocytes at day 1 and day 8 (24 hours post sargramostim dose on both time points) in all subjects. Arrows indicate the days sargramostim was administered to the subjects.

### DETAILED DESCRIPTION

The present disclosure relates to, in part, the finding that GM-CSF can be used in combination with an immunotherapeutic agent as an effective treatment for specific cancer subjects, selected using HLA-DR as a predictive marker of non-responsiveness to monotherapy, e.g., with the immunotherapeutic agent (e.g., an antibody or antibody format directed against a checkpoint marker).

In aspects, the present disclosure relates to improved cancer treatments with an immunotherapeutic agent e.g., checkpoint inhibiting agent (e.g., an antibody or antibody format directed against a checkpoint marker) in a subject that has failed, is intolerant, is resistant, or is refractory to the immunotherapeutic agent e.g., checkpoint inhibiting agent (e.g., an antibody or antibody format directed against a checkpoint marker). For instance, in embodiments, evaluation of the presence, absence or level of HLA-DR informs or predicts the subject's response, and, without limitation, the administration of GM-CSF converts the subject that has failed, is intolerant, is resistant, or is refractory to the immunotherapeutic agent e.g., checkpoint inhibiting agent (e.g., an antibody or antibody format directed against a checkpoint marker) to a responder. In embodiments, the GM-CSF modulates HLA-DR, or cells expressing the same, to improve a subject's responsiveness to the immunotherapeutic agent e.g., checkpoint inhibiting agent (e.g., an antibody or antibody format directed against a checkpoint marker).

Accordingly, in aspects, the disclosure provides methods for treating cancer.

### Compositions of Immunotherapeutic/Immunoregulatory Agents

In embodiments, the immunoregulatory agent is an antibody or antibody format which is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

In embodiments, the immunoregulatory agent is an antibody or antibody format directed against a checkpoint marker.

In embodiments, the immunoregulatory agent is an antibody or antibody format is directed against PD-1 or PD-L1. In embodiments, the anti-PD-1 and/or the anti-PD-L1 agent is an antibody or an antibody format. In other embodiments, the inhibitory immunoregulatory agent is a component of the PD-1/PD-L1 signaling pathway. In further embodiments, the agent disrupts the interaction between PD-1 and PD-L1.

In embodiments, the antagonist of PD-1 is an antibody, for example, an antibody which binds to the extracellular domain of PD-1 and blocks the inhibitory PD-1 signaling. In various embodiments, the antagonist of PD-1 can bind PD-1 on exhausted T cells or a tumor cell. In other embodiments, the antagonist of PD-1 is an agent that induces proliferation, activation and/or activity of effector T cells. In embodiments, the anti-PD-1 antibody is selected from nivolumab, pembrolizumab and pidilizumab.

In embodiments, the antagonist of PD-1 signaling is an anti-PD-L1 antibody. In various embodiments, the antagonist of PD-L1 can bind PD-L1 on tumor cells or antigen-presenting cells. In other embodiments, the antagonist of PD-L1 is an agent that sensitizes tumor cells to cell killing. In embodiments, the anti-PD-L1 antibody is selected from atezolizumab, avelumab, durvalumab and BMS-936559.

In embodiments, the immunotherapeutic agent is an anti-CTLA-4 antibody. In other embodiments, the inhibitory immunoregulatory agent is a component of the CTLA-4 signaling pathway. In further embodiments, the agent disrupts the interaction between CTLA-4 and its ligands, CD80 and CD86. In embodiments, the anti-CTLA-4 antibody is selected from ipilimumab, tremelimumab, AGEN1884, and RG2077.

In embodiments, the immunoregulatory agent is selected from PD-L2, Tim-3, and LAG-3. In further embodiments, the agents that modulate one of PD-L2, Tim-3, and LAG-3 is an antibody or antibody format specific for one of PD-L2, Tim-3, and LAG-3.

### Compositions of GM-CSF

GM-CSF used in the practice of the disclosure includes any pharmaceutically safe and effective GM-CSF, or any derivative thereof having the biological activity of GM-CSF. In an embodiment, the GM-CSF used in the practice of the present methods is rhu GM-CSF, such as sargramostim (LEUKINE). Sargramostim is a biosynthetic, yeast-derived, recombinant human GM-CSF, having a single 127 amino acid glycoprotein that differs from endogenous human GM-CSF by having a leucine instead of a proline at position 23. Other natural and synthetic GM-CSFs, and derivatives thereof having the biological activity of natural human GM-CSF, may be equally useful in the practice of the disclosure.

In embodiments, the GM-CSF is produced or producible in bacteria, yeasts, plants, insect cells, and mammalian cells. In embodiments, the GM-CSF is produced or producible in Escherichia coli cells. In embodiments, the GM-CSF is produced or producible in yeast cells. In embodiments, the GM-CSF is produced or producible in Chinese hamster ovary cells (CHO). In embodiments, the GM-CSF is not produced in E. coli cells. In embodiments, the GM-CSF is produced in a cell that allows for glycosylation, e.g., yeast or CHO cells.

In embodiments, the GM-CSF has an amino acid sequence of SEQ ID NO: 1, or a variant of at least about 90%, or at least about 93%, or at least about 95%, or at least about 97%, or at least about 98% identity thereto. In embodiments, the GM-CSF has an amino acid sequence of SEQ ID NO: 2, or a variant of at least about 90%, or at least about 93%, or at least about 95%, or at least about 97%, or at least about 98% identity thereto. In embodiments, the GM-CSF is one of molgramostim, sargramostim, and regramostim.

Without wishing to be bound by theory, the core of hGM-CSF is comprised of four helices that pack at angles. Crystal structures and mutagenic analysis of rhGM-CSF (Rozwarski D A et al., Proteins 26:304-13, 1996) showed that, in addition to apolar side chains in the protein core, 10 buried hydrogen bonding residues involve intramolecular hydrogen bonding to main chain atoms that were better conserved than residues hydrogen bonding to other side chain atoms; 24 solvation sites were observed at equivalent positions in the two molecules in the asymmetric unit, and the strongest among these was located in clefts between secondary structural elements. Two surface clusters of hydrophobic side chains are located near the expected receptor binding regions. Mutagenesis of residues on the helix A/helix C face confirmed the importance of certain Glu, Gly, and Gln residues. These residues are therefore not to be substituted in the functional substitution variants of hGM-CSF for use in the present disclosure and these helices are to be retained in a functional fragments or deletion variants of hGM-CSF for use in this disclosure. Further, in embodiments, one of ordinary skill can reference UniProtKB entry P04141 for structure information to inform the identity of variants.

The N-terminal helix of hGM-CSF governs high affinity binding to its receptor (Shanafelt A B et al., EMBO J 10:4105-12, 1991) Transduction of the biological effects of GM-CSF requires interaction with at least two cell surface receptor components, (one of which is shared with the cytokine IL-5). The above study identified receptor binding determinants in GM-CSF by locating unique receptor binding domains on a series of human-mouse hybrid GM-CSF cytokines. The interaction of GM-CSF with the shared subunit of their high affinity receptor complexes was governed by a very small part of the peptide chains. The presence of a few key residues in the N-terminal α-helix of was sufficient to confer specificity to the interaction.

In embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids (e.g., selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Modification of the amino acid sequences may be achieved using any known technique in the art e.g., site-directed mutagenesis or PCR based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N. Y., 1989. Without wishing to be bound by theory, the degree of glycosylation of biosynthetic GM-CSFs appears to influence half-life, distribution, and elimination. (Lieschke and Burgess, N. Engl. J. Med. 327:28-35, 1992; Dorr, R. T., Clin. Ther. 15:19-29, 1993; Horgaard et al., Eur. J. Hematol. 50:32-36, 1993). In embodiments, the present GM-CSF molecules are glycosylated.

### Biomarker

In aspects, the present methods relate to the conversion of a checkpoint inhibitor poorly responsive or intolerant cancer subject to a checkpoint inhibitor responsive or tolerant one.

In aspects, the present disclosure relates to a method of treating a subject in need of therapy wherein the subject is characterized by having failed or being intolerant or refractory to a treatment with an immunomodulatory agent, such as a checkpoint inhibitor. In other aspects, an assessment of the subject having failed or being intolerant or refractory to a treatment with a checkpoint inhibitor comprises measuring of a biomarker in a sample of the subject.

In embodiments, an assessment of the subject having failed or being intolerant or refractory to a treatment with a checkpoint inhibitor comprises measuring a variety of subject parameters. In embodiments, surgical specimens may be analyzed using, e.g., immunohistochemical or immunofluorescence techniques may be used to evaluate tumor antigen expression, T cell infiltrate, tumor necrosis, and/or expression of surface markers such as PD-L1. In embodiments, the tumor microenvironment can be dissected histopathologically to characterize spatial relationships between tumor and immune infiltrate. In embodiments, transcriptional profiling assays can evaluate changes in gene expression in both the tumor and in lymphocytes. In embodiments, enzyme-linked immunoabsorbent assays or protein arrays can assess treatment-related production of tumor-specific antibodies in the serum. In embodiments, flow cytometric analysis of tumor-infiltrating lymphocytes (TILs) and/or peripheral blood mononuclear cells (PBMCs) can quantitate the effect of therapy on immune subsets such as, e.g., CD25+T regs, activated CD8+ T cells, or myeloid-derived suppressor cells. In embodiments, polychromatic flow cytometry can be used to measure multiple surface and intracellular markers, allowing characterization of T cell phenotype and activation state. In embodiments, whole blood can be used to evaluate changes in cell count and/or cell ratios with therapy and/or changes in cytokine levels. In embodiments, deep sequencing techniques re used to yield quantification of changes in individual T cell clonotypes.

In embodiments, an assessment of the subject having failed or being intolerant or refractory to a treatment with a checkpoint inhibitor comprises measuring the presence, absence, or amount of Human Leukocyte Antigen-DR (HLA-DR) isotype in a sample of the subject.

In embodiments, the present disclosure relates to a method for treating cancer wherein HLA-DR is used as a biomarker for predicting or determining the therapeutic efficacy of a checkpoint inhibitor therapy.

In embodiments, the presence, absence or amount of HLA-DR by detection of protein and/or nucleic acids in a sample of the subject.

In embodiments, the presence, absence or amount of HLA-DR is determined by ELISA, immunohistochemical staining, western blotting, in-cell western, immunofluorescent staining, or fluorescent activating cell sorting (FACS) in a sample of the subject.

In embodiments, the method for determining the presence, absence or amount of HLA-DR is a method of characterizing a subject or selecting a subject for the treatment comprising GM-CSF and/or checkpoint inhibitor agents.

In embodiments, the method of determining HLA-DR presence, absence or levels for the purposes of subject selection employs a sample, the sample selected from a biopsy, tissue or body fluid.

In embodiments, the subject is treated by modulating and/or increasing the expression of HLA-DR on monocytes.

In embodiments, the subject is treated by modulating immune cell counts and/or cell ratios and/or modulating immune cell function and/or modulating immune cell phenotype and characteristics and/or changes in cytokine levels.

In embodiments, the method of subject selection is undertaken using a sample of the subject, where the sample is selected from blood, a frozen tumor tissue specimen, cultured cells, circulating tumor cells, and a formalin-fixed paraffin-embedded tumor tissue specimen.

In embodiments, the present methods direct subject treatment decisions. For instance, in embodiments, the methods may detect a low or absent HLA-DR level, and this is correlative with the subject having failed or being intolerant or refractory to a treatment with a checkpoint inhibitor. In such embodiments, without limitation, this directs treatment of the subject with GM-CSF agents, as described herein, to improve response to a checkpoint inhibitor (e.g., by modulating HLA-DR levels). In embodiments, the subject receives, e.g., a greater dose of the checkpoint inhibitor, a longer or more frequent regiment of the checkpoint inhibitor, and/or additional anti-cancer therapies.

In embodiments, the GM-CSF agents, as described herein, potentiate treatment with a checkpoint inhibitor. In embodiments, GM-CSF agents, as described herein, are used as neoadjuvants to the checkpoint inhibitor therapy, e.g., to modulate the subject's immune system before checkpoint inhibitor therapy to be more responsive, e.g., by increasing levels of HLA-DR. In embodiments, GM-CSF agents, as described herein, are used as adjuvants to the checkpoint inhibitor therapy, e.g., to modulate the subject's immune system during or after checkpoint inhibitor therapy to be more responsive, e.g., by increasing levels of HLA-DR.

In embodiments, the present methods direct a subject's treatment to alternatives to checkpoint inhibitor therapy, e.g., as the methods indicate a durable failure, intolerant or refractory state. For instance, in embodiments, the subject may not respond to the GM-CSF treatments and/or may not be capable of HLA-DR modulation and therefore treatment is directed to an alternative therapy (e.g., chemotherapy, radiation, surgery) instead of checkpoint inhibitor therapy. In embodiments, the finding of a durable failure, intolerant or refractory state directs away from checkpoint inhibitor therapy to spare the subject of side effects. In embodiments, the finding of a durable failure, intolerant or refractory state directs away from checkpoint inhibitor therapy and to palliative care.

### Methods of Treatment

In aspects, the present disclosure relates to a method for treating cancer, comprising: administering an effective amount of a composition GM-CSF in conjunction with additional agent(s), for example an immunotherapy agent, such as a checkpoint inhibitor to a subject in need thereof.

In another aspect, the present disclosure relates to a method for treating cancer, comprising: administering an effective amount of a composition comprising GM-CSF in conjunction with an immunotherapeutic agent to a subject in need thereof, wherein the subject is characterized by the presence, absence or amount of HLA-DR isotype in a sample of the subject.

In some aspects, the present disclosure relates to methods for treating cancer, comprising: (i) identifying a subject in need thereof, wherein the subject is characterized by undergoing or having undergone treatment with a checkpoint inhibiting agent and presenting as failed, intolerant, resistant, or refractory to the treatment with a checkpoint inhibiting agent; (ii) determining the presence, absence or amount of HLA-DR isotype in a sample of the subject administering an effective amount of a composition; and (iii) administering an effective amount of a composition comprising GM-CSF.

In further aspects, the present disclosure relates to methods of treating a checkpoint inhibitor treatment-resistant or treatment-refractory cancer comprising: selecting the subject in need thereof wherein the subject is selected based on the presence, absence or amount of HLA-DR isotype in a sample of the subject; and administering an effective amount of a composition comprising GM-CSF in conjunction with an immunotherapeutic agent.

In embodiments, the present disclosure relates to a method for treating cancer, comprising: administering an effective amount of a composition comprising GM-CSF in conjunction with an immunotherapeutic agent to a subject in need thereof, wherein the subject is characterized by as a partial responder or a non-responder.

In embodiments, the present disclosure relates to a method for treating cancer, comprising: administering an effective amount of a composition comprising GM-CSF in conjunction with an immunotherapeutic agent to a subject in need thereof, wherein the subject is characterized by having failed or being intolerant or refractory to a treatment with a checkpoint inhibitor.

In embodiments, the method of treatment causes a non-responder or a partial responder to become a full responder to a therapy against cancer. In embodiments, the method of treatment causes an increase in the survival, proliferation and activation of neutrophils, macrophages, dendritic cells, T cells and/or B cells in a non-responder or a partial responder.

In embodiments, the agent that stimulates the survival, proliferation and activation of neutrophils, macrophages and/or dendritic cells is administered at a time selected from (i) the same time as an immunotherapeutic agent, for example, anti-PD-1; (ii) within about 1 hour, about 2 hours, about 4 hours, about 8 hours, about 12 hours, about 24 hours, about 36 hours, about 48 hours, about 72 hours, or about 96 hours or about 1 week or about 2 weeks following administration of said immunotherapeutic agent; (iii) at least about 1 hour, about 2 hours, about 4 hours, about 8 hours, about 12 hours, about 24 hours, about 48 hours, about 36 hours, about 72 hours, or about 96 hours, or about 1 week or about 2 weeks prior to administration of the immunotherapeutic agent; and/or (iv) after at least an about 10%, about 20%, about 30%, about 40% or about 50% decrease in expression of an extracellular marker such as HLA-DR.

In other embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

### Pharmaceutically Acceptable Salts and Excipients

The compositions described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present disclosure having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N, N-dimethyl-N-(2-hydroxyethyl)amine or tri-(2-hydroxyethyl)amine; N-methyl-D-glucamine; and amino acids such as arginine, lysine, and the like.

In embodiments, the compositions described herein are in the form of a pharmaceutically acceptable salt.

### Pharmaceutical Compositions and Formulations

In various embodiments, the present disclosure pertains to pharmaceutical compositions comprising the compositions, e.g., GM-CSF and/or an additional therapeutic agent, e.g., the immunotherapeutic agent, e.g., checkpoint inhibiting agent described herein, and a pharmaceutically acceptable carrier or excipient.

In embodiments, the additional therapeutic agent is one or more of the checkpoint inhibiting agents described herein.

In embodiments, the additional therapeutic agent is a chemotherapy.

In embodiments, the additional therapeutic agent comprises alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33:183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation and pharmaceutically acceptable salts, acids or derivatives of any of the above.

Any pharmaceutical compositions described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

In various embodiments, pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

The present disclosure includes the described pharmaceutical compositions (and/or additional therapeutic agents) in various formulations. Any inventive pharmaceutical composition (and/or additional therapeutic agents) described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use. In one embodiment, the composition is in the form of a capsule. In another embodiment, the composition is in the form of a tablet. In yet another embodiment, the pharmaceutical composition is formulated in the form of a soft-gel capsule. In a further embodiment, the pharmaceutical composition is formulated in the form of a gelatin capsule. In yet another embodiment, the pharmaceutical composition is formulated as a liquid.

Where necessary, the inventive pharmaceutical compositions (and/or additional therapeutic agents) can also include a solubilizing agent. Also, the agents can be delivered with a suitable vehicle or delivery device as known in the art. Combination therapies outlined herein can be co-delivered in a single delivery vehicle or delivery device.

The formulations comprising the inventive pharmaceutical compositions (and/or additional therapeutic agents) of the present disclosure may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In various embodiments, any pharmaceutical compositions (and/or additional therapeutic agents) described herein is formulated in accordance with routine procedures as a composition adapted for a mode of administration described herein.

Routes of administration include, for example: oral, intradermal, intramuscular, intraperitoneal, intravenous, subcutaneous, intranasal, epidural, sublingual, intranasal, intracerebral, intravaginal, transdermal, rectally, by inhalation, or topically. Administration can be local or systemic. In embodiments, the administering is effected orally. In another embodiment, the administration is by parenteral injection. The mode of administration can be left to the discretion of the practitioner and depends in-part upon the site of the medical condition. In most instances, administration results in the release of any agent described herein into the bloodstream.

In specific embodiments, the GM-CSF (and/or additional therapeutic agents) is administered via an intravenous (IV) route, subcutaneous (SC) route, or inhalation (IH) route of administration.

In specific embodiments, the GM-CSF (and/or additional therapeutic agents) is administered to the lung.

In specific embodiments, the GM-CSF (and/or additional therapeutic agents) is administered via aerosol or nebulizer.

In specific embodiments, the aerosol or nebulizer is selected from liquid nebulization, dry powder dispersion and meter-dose administration. In specific embodiments, the aerosol or nebulizer is selected from jet flow or mesh vibrating.

In specific embodiments, the GM-CSF (and/or additional therapeutic agents) is administered by inhalation. Without wishing to be bound by theory, inhalation of sargramostim produces low systemic exposure in the subject.

In one embodiment, the pharmaceutical compositions (and/or additional therapeutic agents) described herein are formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any pharmaceutical compositions (and/or additional therapeutic agents) described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Dosage forms suitable for parenteral administration (e.g., intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage and should be preserved against microorganisms. The carrier can be a solvent or dispersion medium containing, for example, water, ethanol, polyol (for example, glycerol, propylene glycol, and liquid polyetheylene glycol), and suitable mixtures thereof.

The compositions provided herein, alone or in combination with other suitable components, can be made into aerosol formulations (i.e., “nebulized”) to be administered via inhalation. Aerosol formulations can be placed into pressurized acceptable propellants, such as dichlorodifluoromethane, propane, nitrogen, and the like.

Any inventive pharmaceutical compositions (and/or additional therapeutic agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropyl cellulose, hydropropylmethyl cellulose, polyvinylpyrrolidone, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The disclosure thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

### Administration and Dosage

It will be appreciated that the actual dose of the composition to be administered according to the present disclosure will vary according to the particular dosage form, and the mode of administration. Many factors that may modify the action of the composition (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

In embodiments, the GM-CSF is administered at a total dose of about 125 μg, about 150 μg, or about 200 μg, or about 250 μg, or about 300 μg, or about 350 μg.

In embodiments, the GM-CSF is administered at a dose of about 125 μg, about 150 μg, or about 200 μg, or about 250 μg, or about 300 μg, or about 350 μg.

In embodiments, the GM-CSF is administered once or twice daily for about 1 day or about 2 days, or about 3 days, or about 4 days, or about 5 days, or about 6 days, or about 7 days or about 8 days.

### Combination Therapy and Additional Therapeutic Agents

In various embodiments, the pharmaceutical composition of the present disclosure is co-administered in conjunction with additional agent(s), for example an immunotherapy agent, such as a checkpoint inhibitor. Co-administration can be simultaneous or sequential.

In one embodiment, the additional immunotherapeutic agent and the GM-CSF of the present disclosure are administered to a subject simultaneously. The term “simultaneously” as used herein, means that the immunotherapeutic agent and the GM-CSF are administered with a time separation of no more than about 60 minutes, such as no more than about 30 minutes, no more than about 20 minutes, no more than about 10 minutes, no more than about 5 minutes, or no more than about 1 minute. Administration of the immunotherapeutic agent and the GM-CSF can be by simultaneous administration of a single formulation (e.g., a formulation comprising the additional therapeutic agent and the GM-CSF composition) or of separate formulations (e.g., a first formulation including the immunotherapeutic agent and a second formulation including the GM-CSF composition).

Co-administration does not require the therapeutic agents to be administered simultaneously, if the timing of their administration is such that the pharmacological activities of the immunotherapeutic agent and the GM-CSF overlap in time, thereby exerting a combined therapeutic effect. For example, the immunotherapeutic agent and the targeting moiety, the GM-CSF composition can be administered sequentially. The term “sequentially” as used herein means that the immunotherapeutic agent and the GM-CSF are administered with a time separation of more than about 60 minutes. For example, the time between the sequential administration of the immunotherapeutic agent and the GM-CSF can be more than about 60 minutes, more than about 2 hours, more than about 5 hours, more than about 10 hours, more than about 1 day, more than about 2 days, more than about 3 days, more than about 1 week apart, more than about 2 weeks apart, or more than about one month apart. The optimal administration times will depend on the rates of metabolism, excretion, and/or the pharmacodynamic activity of the additional therapeutic agent and the GM-CSF being administered. Either the immunotherapeutic agent or the GM-CSF composition may be administered first.

Co-administration also does not require the therapeutic agents to be administered to the subject by the same route of administration. Rather, each therapeutic agent can be administered by any appropriate route, for example, parenterally or non-parenterally.

In embodiments, the GM-CSF described herein acts synergistically when co-administered with the immunotherapeutic agent. In such embodiments, the targeting moiety, the GM-CSF composition and the immunotherapeutic agent may be administered at doses that are lower than the doses employed when the agents are used in the context of monotherapy.

### Sequences

SEQ ID NO: 1 is wild type GM-CSF:
APARSPSPSTQPWEHVNAIQEARRLLNLSRDTAAEMNETVEVISEMFDL
QEPTCLQTRLELYKQGLRGSLTKLKGPLTMMASHYKQHCPPTPETSCAT
QIITFESFKENLKDFLLVIPFDCWEPVQE
SEQ ID NO: 2 is sargramostim:
APARSPSPSTQPWEHVNAIQEALRLLNLSRDTAAEMNETVEVISEMFDL
QEPTCLQTRLELYKQGLRGSLTKLKGPLTMMASHYKQHCPPTPETSCAT
QIITFESFKENLKDFLLVIPFDCWEPVQE

### Definitions

The following definitions are used in connection with the disclosure disclosed herein. Unless defined otherwise, all technical and scientific terms used herein have the same meaning as commonly understood to one of skill in the art to which this disclosure belongs.

A “subject” includes an animal, including a mammal, e.g., a human, mouse, rat, guinea pig, dog, cat, horse, cow, pig, rabbit, sheep, or non-human primate, such as a monkey, chimpanzee, or baboon. A “subject” also includes a non-mammal, such, for example, a zebrafish. In embodiments, the subject and/or animal may comprise fluorescently-tagged cells (with e.g., GFP). In embodiments, the subject and/or animal is a transgenic animal comprising a fluorescent cell. In embodiments, the human is a pediatric human. In other embodiments, the human is an adult human. In other embodiments, the human is a geriatric human. In other embodiments, the human may be referred to as a patient.

As used herein, “a,” “an,” or “the” can mean one or more than one. Further, the term “about” when used in connection with a referenced numeric indication means the referenced numeric indication plus or minus up to 10% of that referenced numeric indication. For example, the language “about 50” covers the range of 45 to 55.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present disclosure, or embodiments thereof, may alternatively be described using alternative terms such as “consisting of” or “consisting essentially of.”

### EXAMPLES

### Example 1: Expression and Kinetics of HLA-DR on Human Monocytes

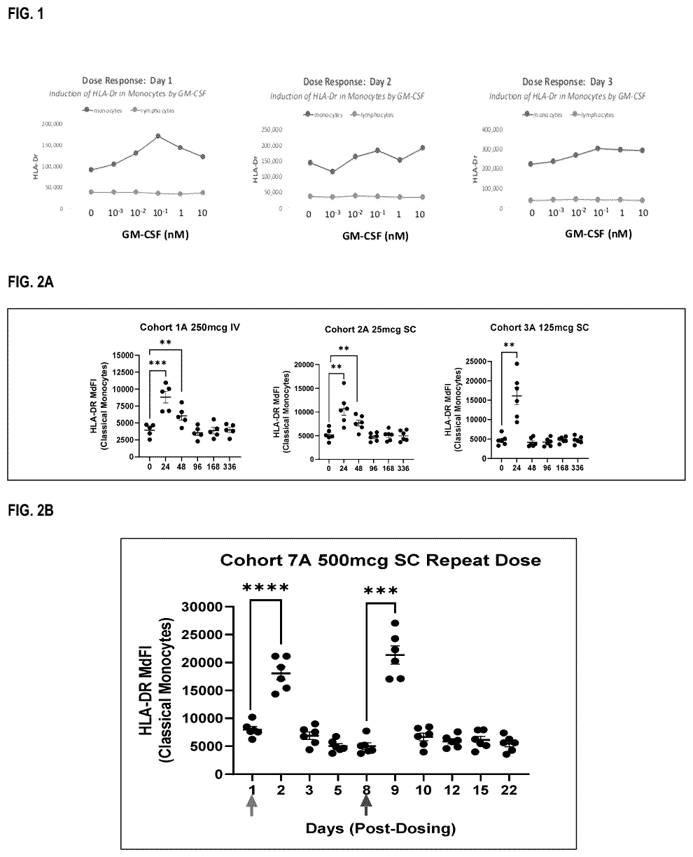

Blood was collected in healthy volunteers, and a whole blood stain, lyse-no-wash protocol was used to prepare stained cell samples for analysis. Human monocytes and lymphocytes from the donors were incubated with LEUKINE at various concentrations from 0.001 nM to 10 nM. The expression of HLA-DR on monocytes and lymphocytes was measured on day 1, day 2 and day 3 following treatment. Treatment with LEUKINE enhanced the kinetics of HLA-DR on monocytes but not lymphocytes (FIG. 1).

### Example 2: Pharmacokinetics, Pharmacodynamics, and Expression of Sargramostim Administered Subcutaneously, Intravenously, or by Inhalation

In a single center, single ascending dose (SAD) and repeat dose study in healthy subjects that comprised of 2 parts, 6 subjects each were dosed in 7 cohorts, with different dose levels of sargramostim by intravenous (IV) infusion, subcutaneous (SC) injection, or inhalation (IH) administration as shown in Table 1.

TABLE 1

| Cohort | Route | Dose (micrograms) | Dosing Days |
|---|---|---|---|
| 1A | IV | 250 | Day 1 |
| 2A | SC | 25 | Day 1 |
| 3A | SC | 125 | Day 1 |
| 4A | SC | 250 | Day 1 |
| 5A | SC | 500 | Day 1 |
| 6A | IH (Inhaled) | 250 | Day 1 |
| 7A | SC | 500 | Day 1 & Day 8 |

Table 1 illustrates the dose and dosing schedule/regimen for 6 subjects in the single ascending dose (SAD) and repeat dose study in healthy subjects where subjects were given different dose levels of sargramostim by intravenous (IV) infusion, subcutaneous (SC) injection, or inhalation (IH) administration.

Blood samples for pharmacodynamic assessment were collected before and up to 14 days after drug administration, as well as before the first and up to 14 days after the second drug administration.

The expression of HLA-DR was assessed via flow cytometry on classical monocytes (CD14+CD16+) at the indicated timepoints. Treatment with sargramostim enhanced expression of HLA-DR on monocytes at various doses administered and the increases were observed with all administration routes in all subjects in the SAD part of the study (FIG. 2A).

In the repeat dose part of study, increase of HLA-DR expression on classical monocytes was observed at day 1 and day 8 (24 hours post sargramostim dose on both time points) in all subjects. Arrows in FIG. 2B indicate the days sargramostim was administered to the subjects.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

As used herein, all headings are simply for organization and are not intended to limit the disclosure in any manner. The content of any individual section may be equally applicable to all sections.

What is claimed is:

1. A method of selecting a subject for treatment with a checkpoint inhibiting agent, comprising:
(a) determining the presence, absence or amount of Human Leukocyte Antigen-DR isotype (HLA-DR) in a sample from the subject;
(b) administering an effective amount of a granulocyte-macrophage colony-stimulating factor (GM-CSF) agent to the subject demonstrating an absence or low level of HLA-DR; and
(c) administering an effective amount of a checkpoint inhibiting agent.

2. A method for treating a cancer, comprising:
(a) identifying a subject undergoing or having undergone treatment with a checkpoint inhibiting agent and presenting as failed, intolerant, resistant, or refractory to the treatment with a checkpoint inhibiting agent;
(b) determining the presence, absence or amount of Human Leukocyte Antigen-DR isotype (HLA-DR) in a sample from the subject; and
(c) administering an effective amount of a GM-CSF agent to the subject demonstrating an absence or low level of HLA-DR.

3. A method for treating a checkpoint inhibitor treatment-resistant or treatment-refractory cancer, comprising:
(a) selecting a subject afflicted with cancer for treatment;
(b) administering an effective amount of a GM-CSF agent to the subject; and
(c) administering an effective amount of a checkpoint inhibiting agent to the subject,
wherein the subject is selected based on a presence, absence or amount of HLA-DR in a sample from the subject.

4. The method of any of claims 1-3, wherein the checkpoint inhibiting agent is an agent that modulates one or more PD-1, PD-L1, PD-L2, CTLA-4, Tim-3, and LAG-3.

5. The method of any of claims 1-3, wherein the agent that modulates one of PD-1, PD-L1, PD-L2, CTLA-4, Tim-3, and LAG-3 is an antibody or antibody format specific for one of PD-1, PD-L1, PD-L2, CTLA-4, Tim-3, and LAG-3.

6. The method of any one of claims 1-5, wherein the subject is identified as a complete responder, a partial responder or a non-responder.

7. The method of any one of claim 1-6, wherein the agent is an antibody or an antibody format.

8. The method of claim 7, wherein the antibody or antibody format is selected from one or more of a monoclonal antibody, polyclonal antibody, antibody fragment, Fab, Fab′, Fab′-SH, F(ab′)2, Fv, single chain Fv, diabody, linear antibody, bispecific antibody, multispecific antibody, chimeric antibody, humanized antibody, human antibody, and fusion protein comprising the antigen-binding portion of an antibody.

9. The method of claim 7, wherein the antibody or antibody format specific for PD-1 is selected from nivolumab, pembrolizumab, and pidilizumab.

10. The method of claim 7, wherein the antibody or antibody format specific for PD-L1 is selected from atezolizumab, avelumab, durvalumab, and BMS-936559.

11. The method of claim 7, wherein the antibody or antibody format specific for CTLA-4 is selected from ipilimumab, tremelimumab, AGEN1884, and RG2077.

12. The method of any one of claims 1-11, wherein the presence, absence or amount of HLA-DR is determined by detection of protein and/or nucleic acids.

13. The method of claim 12, wherein the presence, absence or amount of HLA-DR is determined by ELISA, immunohistochemical staining, western blotting, in-cell western, immunofluorescent staining, or fluorescent activating cell sorting (FACS).

14. The method of claim 1 or 3, wherein HLA-DR is used as a biomarker for predicting or determining the therapeutic efficacy of a checkpoint inhibitor therapy.

15. The method of any one of claims 1-14, wherein the sample is a biopsy, tissue or body fluid.

16. The method of any one of claims 1-15, wherein the sample is selected from blood, a frozen tumor tissue specimen, cultured cells, circulating tumor cells, and a formalin-fixed paraffin-embedded tumor tissue specimen.

17. The method of claim 16, wherein the sample is blood.

18. The method of any one of claims 1-17, wherein the GM-CSF agent has an amino acid sequence of SEQ ID NO: 1, or a variant of at least about 90%, or at least about 93%, or at least about 95%, or at least about 97%, or at least about 98% identity thereto.

19. The method of any one of claims 1-17, wherein the GM-CSF agent has an amino acid sequence of SEQ ID NO: 2, or a variant of at least about 90%, or at least about 93%, or at least about 95%, or at least about 97%, or at least about 98% identity thereto.

20. The method of any one of claims 1-17, wherein the GM-CSF agent is one of molgramostim, sargramostim, and regramostim.

21. The method of any one of claims 1-17, wherein the GM-CSF agent is sargramostim.

22. The method of any one of claims 18-21, wherein the GM-CSF is administered at a total dose of about 125 μg, about 150 μg, or about 200 μg, or about 250 μg, or about 300 μg, or about 350 μg.

23. The method of any one of claims 18-21, wherein the GM-CSF is administered at a dose of about 125 μg, about 150 μg, or about 200 μg, or about 250 μg, or about 300 μg, or about 350 μg.

24. The method of any one of claims 22-23, wherein the GM-CSF is administered once or twice daily for about 1 day or about 2 days, or about 3 days, or about 4 days, or about 5 days, or about 6 days, or about 7 days or about 8 days.

25. The method of any one of claims 1-24, wherein the GM-CSF is administered via an intravenous (IV) route, subcutaneous (SC) route, or inhalation (IH) route of administration.

26. The method of any of the above claims, wherein the subject is treated by modulating clonal expansion, survival, differentiation, non-proliferative maturation effects, and activation state of hematopoietic progenitor cells.

27. The method of any of the above claims, wherein the subject is treated by modulating hematopoietic progenitor cells, by stimulating the survival, proliferation, non-proliferative maturation effects, and activation of neutrophils, macrophages and/or dendritic cells.

28. The method of any of the above claims, wherein the subject is treated by modulating and/or increasing the expression of HLA-DR on monocytes.

29. The method of any of the above claims, where the subject is treated by modulating immune cell counts and/or cell ratios and/or modulating immune cell function and/or modulating immune cell phenotype and characteristics and/or changes in cytokine levels.

30. The method of any of the above claims, wherein the subject is treated following cancer therapy by modulating hematopoietic progenitor cells, by stimulating the survival, proliferation and activation of neutrophils, macrophages and/or dendritic cells.

31. A method of any of the above claims, comprising administering a therapy to a non-responder or a partial responder a therapy that increases the survival, proliferation and activation of neutrophils, macrophages, dendritic cells, T cells and/or B cells.

32. A method of any of the above claims, wherein a non-responder becomes a responder to a therapy against cancer.

33. A method of any of the above claims, wherein a partial responder becomes a responder to a therapy against cancer.

34. The method of any one of the above claims, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; leukemia; liver cancer; lung cancer; small-cell lung cancer, non-small cell lung cancer, adenocarcinoma of the lung squamous carcinoma of the lung; melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.


SEQUENCE LISTING (2 sequences)

SEQ ID NO: 1 - protein, 127 aa, synthetic construct [sequence omitted]

SEQ ID NO: 2 - protein, 127 aa, synthetic construct
APARSPSPSTQPWEHVNAIQEALRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE